(19) United States
(12) Patent Application Publication
(10) Pub. No.: US 2017/0354764 A1
(43) Pub. Date: Dec. 14, 2017

(54) COATED BALLOON CATHETER AND COMPOSITION FOR COATING SAID BALLOON CATHETER

(71) Applicant: DOT GMBH, Rostock (DE)

(72) Inventors: Hans-Georg NEUMANN, Rostock (DE); Mischa BUHRMEISTER, Papendorf (DE)

(21) Appl. No.: 15/596,231

(22) Filed: May 16, 2017

(30) Foreign Application Priority Data
Jun. 13, 2016 (DE) 10 2016 110 815.4

(51) Int. Cl. A61L 29/08; A61L 29/16; A61M 25/10; A61K 31/337

(52) U.S. Cl. CPC A61L 29/085; A61K 31/337; A61L 29/16; A61M 25/1029; A61L 2420/02; A61L 2420/06; A61M 2025/1031; A61M 2025/105; A61L 2300/416

(57) ABSTRACT

The invention relates to a coated balloon catheter with a catheter substrate and a coating on the catheter substrate. The coating comprises a pharmaceutically active ingredient embedded in a binder matrix. The binder matrix consists of a polyethylene glycol-polyvinyl alcohol copolymer (PEG-PVA copolymer) and optionally shellac or a shellac derivative and additional pharmaceutically acceptable additives. A composition for coating the balloon catheter comprises the pharmaceutically active ingredient and a binder consisting of a PEG-PVA copolymer and optionally shellac or a shellac derivative. The active ingredient and the binder are dissolved in a solvent consisting of water, DMSO and at least one additional organic solvent indefinitely miscible with water.

### FIELD OF THE INVENTION

The invention relates to a coated balloon catheter and a composition for coating said balloon catheter.

### BACKGROUND OF THE INVENTION

Balloon catheters are used for dilatating pathologically narrowed blood vessels (stenosis). The balloon that is attached to a vascular catheter is inserted via an artery, for example the femoral artery, and advanced to the narrowed site of the vessel under X-ray control. There, the balloon is pressurized and slowly unfolded, thus dilatating the narrowed site and allowing an uninterrupted blood flow.

In addition, a stent can be implanted to prevent renewed stenosis. Depending on the site of stenosis, the size of the vessel and previous diseases drug-coated stents can be used.

Some patients show a new narrowing (restenosis) as early as a few months after dilatation of the stenosis. Restenosis can be attributed to excessive proliferation, in particular of the smooth muscle cells, that is caused by injuries due to forcible dilatation of the vascular walls. After the injuries have healed, proliferation does not stop immediately and thus often leads to restenosis. To prevent such restenosis, drug-coated balloon catheters, so called drug eluting balloons, have been developed. A commonly used active ingredient with an anti-proliferative effect is paclitaxel.

In addition, drug-coated balloon catheters may be used for local administration of drugs to the vascular wall without dilatating the vessels, for example when treating changes in the vascular wall not linked with a stenosis (e.g. vulnerable plaques, attached thrombi) or when treating vessels by mechanical means or thermal methods. In these cases, the balloon does not abut completely with the irregular vascular walls, and the active ingredient can only be transferred at the site of contact. For this reason, a coating of the balloon's surface that is as regular as possible is desired.

Coated balloon catheters are known from WO 2005/089855 A1. WO 2004/028582 A1 discloses multiply folded balloons coated with a composition comprising a pharmacological active ingredient and a contrast agent, preferably within the folds. WO 2004/006976 A1 describes a method for spray coating balloon catheters.

From DE 20 2010 017 248 U1 a balloon catheter is known that has a coating made of paclitaxel and shellac. The use of shellac as a binder is said to promote a rapid release of the active ingredient paclitaxel during the short contact period of the balloon catheter with the vascular wall.

During unfolding of the balloon catheter the contact period of the balloon with the vascular wall at the site of stenosis is only some seconds to a few minutes. Thus, a uniform coating of the balloon's surface is advantageous as it allows the use of the contact surface of the balloon with the vascular wall for administering the active ingredient as completely as possible. At the same time, the coating has to be sufficiently stable to endure the transport through the blood vessels.

With the coated balloon catheters currently approved there is the problem that a substantial part of the coating peels off on the way to its place of destination resulting in an insufficiently amount of the active ingredient reaching its site of action. This problem is often counteracted by applying a multi-layer system onto the balloon containing one or more adhesion layers to improve adhesion on the balloon's surface, the actual active ingredient layer and one or more top coats to reduce the risk of peeling off of the active ingredient layer.

WO 2014/029442 describes such a balloon catheter with a coating that comprises at least one layer with an anti-proliferative, immunosuppressive, anti-angiogenic, anti-inflammatory, anti-restenotic and/or anti-thrombotic active agent and a top coat without any active ingredient made of a polyvinyl alcohol-polyethylene glycol graft copolymer.

However, the presence of a top coat over the layer containing the active ingredient on the balloon's surface can result in an insufficient amount of the active ingredient being administered to the vascular wall, in particular if the balloon's surface is only partly in contact with the vascular wall to be treated. But without the top layer, substantial loss of active ingredient already occurs when inserting the balloon catheter into the vessels.

### SUMMARY OF THE INVENTION

Thus, the object of the invention is to provide a balloon catheter with a stable coating that shows a distribution of the active ingredient on the surface of the balloon catheter that is as homogeneous as possible and allows a controlled administration of the active ingredient to the vascular wall at the site of stenosis.

According to the invention, this object is resolved by a balloon catheter pursuant to claim 1.

Advantageous embodiments of the invention are stated in the sub-claims which can optionally be combined with each other.

The coated balloon catheter comprises a catheter substrate and a coating on the catheter substrate. The coating comprises a pharmaceutically active ingredient embedded in a binder matrix. According to the invention, the binder matrix consists of a polyethylene glycol-polyvinyl alcohol copolymer (PEG-PVA copolymer) and optionally shellac or a shellac derivative. In addition, the coating can comprise additional pharmaceutically acceptable additives.

The use of a binder matrix with a PEG-PVA copolymer content leads to mechanically sufficiently stable coatings from which only some active ingredient peels off during insertion of the catheter into the vessels. Despite this the coatings are such that a sufficiently high amount of the pharmaceutically active ingredient is administered during the short contact periods of the balloon coating with the vascular walls at the site of stenosis.

However, most of the pharmaceutically active ingredients with a proliferative effect are insoluble or only poorly soluble in water, while the PEG-PVA copolymer already used as a protective top coat in the state of the art is only described as being water-soluble. The inventors have now recognized that a stable coating containing PEG-PVA copolymer as a binder and the anti-proliferative active ingredients insoluble in water, such as paclitaxel, can be produced by selectively choosing the solvents used for the coating solution and their sequence in dissolving the binder and the active ingredient. Surprisingly, the structuring of the active ingredient in the coating and thus the availability of the active ingredient at the site of stenosis can also be set and controlled by selecting the solvent.

Thus, another object of the invention is a composition for coating a balloon catheter, the composition comprising a pharmaceutically active ingredient and a binder made of a PEG-PVA copolymer and optionally shellac or a shellac derivative, with the pharmaceutically active ingredient and the binder being dissolved in a mixture of water and at least one additional organic solvent that is fully miscible with water.

In addition, the invention relates to a method for producing a coated balloon catheter comprising the following steps:
a) providing an aqueous solution of a PEG-PVA copolymer and optionally a water-soluble shellac derivative;
b) gradually adding at least one additional organic solvent fully miscible with water to the aqueous solution of step a) to form an aqueous-organic solution;
c) mixing the aqueous-organic solution with a pharmaceutically active ingredient and homogenizing the mixture to form a coating solution containing the active ingredient;
d) optionally adding DMSO in a percentage of up to 10 volume percent to the coating solution containing the active ingredient of step c) relative to the total volume of water and organic solvent; and
e) applying the coating solution containing the active ingredient of step c) or d) onto a surface of the balloon catheter and drying the coating solution to form the coated balloon catheter.

### DESCRIPTION OF PREFERRED EMBODIMENTS

A pharmaceutically active ingredient according to the invention is a pharmacologically active substance, in particular a medicinal product. The pharmaceutically active ingredient is preferably an anti-proliferative, immunosuppressive, anti-inflammatory, anti-phlogistic, anti-hyperplastic, anti-neoplastic, anti-mitotic, cytostatic, cytotoxic, anti-angiogenic, anti-restenotic, microtubule inhibiting, anti-migrative or anti-thrombotic substance. Anti-proliferative active ingredients, in particular paclitaxel and/or rapamycin, immunosuppressive active ingredients such as everolimus, biolimus and/or tacrolimus, cortisone or a combination thereof are preferred.

The pharmaceutically active ingredient is preferably soluble in polar organic solvents such as methanol, ethanol, acetone, ethyl acetate, trichloromethane and DMSO, but insoluble or only slightly soluble in water.

According to a preferred embodiment the PEG-PVA copolymer is a graft copolymer, particularly preferably a PEG-PVA graft copolymer with a PEG content of 15 to 30 mole percent and a PVA content of 70 to 85 mole percent. Such graft copolymers are commercially available and approved as excipients. According to the manufacturers' information the PEG-PVA copolymer is soluble in water and aqueous systems such as weak acids or bases. In a mixture of water and ethanol (ratio: 1:1 (v/v)) up to 25 weight percent of the PEG-PVA copolymer can still be dissolved. The commercially available products can contain additives such as colloidal silicon dioxide in small amounts (less than 1 weight percent).

Preferably, the PEG-PVA copolymer has an average molar mass Mₙ from 30,000 to 60,000 g/mole, more preferably from 40,000 to 50,000 g/mole.

According to another embodiment the binder matrix further contains shellac or a shellac derivative. The shellac derivative is preferably a water-soluble shellac derivative, particularly preferably a water-soluble ammonium salt of shellac. Shellac promotes the release of the active ingredient into the tissue of the blood vessels, in particular during short contact periods, and improves the adhesive strength of the coating on the balloon.

In addition to shellac or shellac derivatives the coating can contain additional pharmaceutically acceptable additives as known to those skilled in the art.

According to a particularly preferred embodiment the binder matrix consists of the PEG-PVA copolymer or of the PEG-PVA copolymer and a water-soluble shellac derivative, for example an ammonium salt of shellac. In this case, the binder matrix does not contain any further additives apart from those already contained in the starting components of the binder matrix, which can be considered as inevitable impurities.

The weight ratio of shellac or the shellac derivative to the PEG-PVA copolymer in the coating is no more than 1:1, preferably no more than 1:2.

Loading of the catheter substrate with the active ingredient is preferably from 0.5 μg/mm² to 10 μg/mm², relative to the outer surface of the balloon when expanded, more preferably from 0.5 μg/mm² to 5 μg/mm² and especially preferably from 1 to 3 μg/mm².

The weight ratio of the active ingredient to the PEG-PVA copolymer in the coating is preferably from 10:1 to 1:2, more preferably from 5:1 to 1:2, even more preferably from 3:1 to 1:2 or from 2:1 to 1:2 and especially preferably from 2:1 to 1:1. If the percentage of the PEG-PVA copolymer is too low, adhesion of the coating on the balloon's surface may be impaired. If the percentage of the PEG-PVA copolymer in the binder matrix is too high, the water solubility of the coating increases and its resistance to peeling off from the balloon's surface decreases as the catheter is transported through the blood vessels.

The entire weight of the coating applied onto the balloon's surface is preferably in a range from 0.75 μg/mm² to 20 μg/mm².

Preferably, the active ingredient is present in the form of particles embedded in the coating, more preferably in the form of nanoscopic and/or microscopic particles and especially preferably in the form of spheroidal and/or needle-like aggregates embedded in the binder matrix. The formation of such aggregates indicates that the phases of the active ingredient and the binder matrix have at least partly separated and, under suitable conditions, microscopic crystals of the active ingredient have already formed. Under the conditions prevailing during transport of the catheter such a coating with particles of the active ingredient embedded in the binder matrix is sufficiently stable in the blood serum. In addition, depending on the case of application, the release rate of the active ingredient at the site of stenosis can be deliberately controlled by the nanoscopic or microscopic structure of the active ingredient particles. The inventors understand that the active ingredient transferred to the tissue in the form of microscopic particles has a lower dissolution rate and can thus remain for a longer period at the site of stenosis, while the active ingredient present in the form of nanoscopic particles has a higher dissolution rate, thus allowing a high initial concentration of the active ingredient.

Nanoscopic or nano-scale particles are those having dimensions from 1 nm to 100 nm. Active ingredient particles having dimensions from 0.1 μm to 100 μm are considered microscopic or micro-scale bodies.

Preferably, the layer containing the active ingredient embedded in the binder matrix is the outermost layer of the coating on the catheter substrate and especially preferably the only layer of the coating. According to an alternative embodiment it can be provided that an adhesive layer containing no active ingredient is applied onto the catheter substrate, over which the layer containing the active ingredient and PEG-PVA copolymer as a binder matrix is then disposed. According to the invention it is not provided that a top layer is applied onto the layer containing the active ingredient.

All commercially available balloons of balloon catheters can be used as a catheter substrate. Balloons with a smooth surface, balloons with grooves or pores and balloons with structured or roughened surfaces are particularly suitable. Further, balloons of catheters provided with folds or wings, in particular so-called multi-fold balloons, are also suitable.

The coated balloon catheter is produced by applying a coating solution onto a surface of the catheter substrate that contains all solid components of the coating. The coating solution can be applied by spray coating, dip coating or printing methods as known by those skilled in the art.

Thus, another object of the invention is a composition for coating a balloon catheter comprising a pharmaceutically active ingredient, in particular an anti-inflammatory active ingredient such as cortisone, an immunosuppressive active ingredient such as everolimus, biolimus and/or tacrolimus or an anti-proliferative active ingredient such as paclitaxel and/or rapamycin and a binder that consists of a PEG-PVA copolymer and optionally shellac or a shellac derivative. The active ingredient and the binder are dissolved in a solvent mixture comprising water and at least one additional organic solvent that is infinitely miscible with water and optionally contains up to 10 volume percent DMSO.

As a pharmaceutical active ingredient, the active ingredients already described can be used. The PEG-PVA copolymer is preferably a PEG-PVA graft copolymer as described above. The shellac derivative is preferably water-soluble and especially preferably a water-soluble ammonium salt of shellac that can be produced in a known manner by reacting shellac with ammonium carbonate. PEG-PVA copolymers and water-soluble shellac derivatives for pharmaceutical and cosmetic applications are commercially available.

According to a preferred embodiment the composition contains up to approximately 5 to 40 volume percent, preferably approximately 5 to 35 volume percent, more preferably 10 to 35 volume percent and especially preferably 15 to 25 volume percent water relative to the content of water and additional organic solvent.

Preferably, the additional organic solvent has a boiling point of less than 100 C. Acetone and lower alcohols such as methanol, ethanol and/or isopropanol are especially preferred, ethanol is particularly preferred. The composition preferably contains approximately 60 to 95 volume percent, more preferably approximately 65 to 95 volume percent, even more preferably 65 to 90 volume percent and particularly preferably 75 to 85 volume percent of the additional organic solvent relative to the content of water and additional organic solvent.

Further, the composition preferably has a DMSO content of additively 0.5 to 10 volume percent, more preferably up to 5 volume percent and especially preferably 2 to 4 volume percent relative to the volume of the mixture of water and additional organic solvent.

The pharmaceutically active ingredient can be present at a concentration of up to 25 mg/ml in the composition. Preferably, the active ingredient concentrations are up to 20 mg or 15 mg per 1 ml solution, more preferably up to 10 mg active ingredient per 1 ml solution. Preferred concentration ranges are 0.5 mg to 20 mg, preferably 1 to 15 mg or 5 mg to 15 mg active ingredient per 1 ml solution.

The weight ratio of the active ingredient to the PEG-PVA copolymer as a binder is preferably 10:1 to 1:2, more preferably 5:1 to 1;2, even more preferably 3:1 to 1:2 or 2:1 to 1:2 and particularly preferably 2:1 to 1:1. If shellac or a shellac derivative is used as an additional binder, the weight ratio of shellac or the shellac derivative to the PEG-PVA copolymer is preferably no more that 1:1, especially preferably no more than 1:2.

The total solid content of the composition can be up to 150 mg/ml, preferably up to 125 mg/ml and particularly preferably up to 100 mg/ml solution. Especially preferably is a total solid content of 1 mg to 100 mg per 1 ml solution, more preferably between 1 mg and 50 mg per 1 ml solution and most preferably of 1 mg to 40 mg or 1 to 30 mg per 1 ml solution.

According to a special embodiment the composition comprises an anti-proliferative and/or immunosuppressive active ingredient, in particular paclitaxel, rapamycin, everolimus, biolimus and/or tacrolimus at a concentration of up to 25 mg/ml and a PEG-PVA copolymer at a concentration of 1 to 50 mg/ml and optionally a water-soluble shellac derivative, especially an ammonium salt of shellac, at a concentration of 0 to 50 mg/ml as a binder. The solvent for the solids consists of a mixture of water, ethanol and/or methanol and optionally DMSO. The water is preferably present in a percentage of 5 to 40 volume percent, more preferably 5 to 35 volume percent relative to the total volume of water and ethanol and/or methanol. DMSO can be added in a percentage of 0.5 to 10 volume percent, preferably 1 to 5 volume percent, more preferably 2 to 4 volume percent relative to the total volume of water and ethanol and/or methanol.

To produce the coating solution the PEG-PVA copolymer and optionally the water-soluble shellac derivative are dissolved in water and the additional organic solvent, for example ethanol, is gradually added. Then, the pharmaceutically active ingredient is added to the aqueous-organic solution of the binder, preferably as a solid, and the mixture is homogenized. Then, DMSO can be added to the solution thus obtained in a percentage of up to 10 volume percent relative to the total volume of water and additional solvent. Optionally, the shellac can be added to the aqueous-ethanolic solution of the PEG-PVA copolymer prior to or after adding the active ingredient.

Surprisingly, if the additional organic solvent, especially ethanol, is added stepwise, the PEG-PVA copolymer continues to be in solution forming a stable, aqueous-ethanolic polymer solution in which the active ingredient can be dissolved homogeneously.

The coating solution can be applied onto the surface of the catheter substrate when the balloon is not expanded or totally or partly expanded. In particular, balloons provided with folds are preferably coated when at least partially expanded.

To improve wetting of the coating solution the balloon's surface can be subjected to a corona treatment or a plasma treatment prior to coating.

After application of the coating solution the balloon's surface is preferably dried in warm air and/or under vacuum; as a result essentially no solvent remains in the coating. If pharmaceutically acceptable liquid or gel forming additives are used, they can remain in the coating.

Finally, the coated balloon can be sterilized and sterilely packed using known procedures such as treatment with ethylene oxide.

By applying the coating solution onto the substrate a homogenous thin liquid film is formed containing the solid components of the coating solution and at least two different solvents having different vapor pressures, preferably at least three different solvents. When the balloon's surface dries water accumulates in the coating solution on the balloon's surface as the organic solvent having the lower boiling point evaporates first, optionally as an azeotrope with water. The active ingredient insoluble in water, preferably paclitaxel, precipitates from the solution forming nanoscopic particles embedded in the dried binder. This step can be identified by the formation of a whitish layer.

As soon as the limit of the binder's solubility in water is exceeded, the binder matrix starts drying as well. The coating seems dry, and a tough, clear to cloudy binder layer is formed enveloping the active ingredient separated from the binder phase and tightly attaching it to the balloon's surface. However, as compared to drying from a water-free solvent, separation of the binder matrix and active ingredient phases takes longer, thus promoting the formation of nanoscopic structures, mostly in the form of spherical or needle-like aggregates of the active ingredient.

The inventors have further recognized that the PEG-PVA copolymer used as a binder is also soluble in DMSO. Thus, adding a small amount of DMSO to the coating solution results in the DMSO still contained in the coating being able to dissolve both the active ingredient, in particular paclitaxel, and the binder matrix made of PEG-PVA copolymer and optionally shellac or the shellac derivative. Thus, depending on the percentage and composition of the binder matrix and the solvent in the coating solution, the active ingredient can precipitate from the binder matrix during the drying process in many ways forming a heterogeneous, adhesive coating with nanoscopic and/or microscopic active ingredient particles separated from the binder matrix. Recrystallization of the binder matrix and the active ingredient can be further controlled by the duration of the drying process.

If the coating solution does not contain DMSO, mainly nanoscopic active ingredient particles which are separated from the binder matrix and embedded in it are formed during the process described above. The addition of DMSO to the coating solution promotes the formation of microscopic structures of the active ingredient particles.

If the DMSO content in the coating solution is such that the amount of DMSO present in the coating during drying of the solvents is not sufficient to completely dissolve the binder matrix and paclitaxel, the drying process is prolonged and paclitaxel can be converted into a microscopic rod or needle structure that is only slightly soluble in water. Thus, the binder phase is separated from the microscopic paclitaxel phase during the very slow drying of DMSO from the coating. The water solubility of the solution is then mainly influenced by the weight ratio of the percentages of the water-soluble binder and the active ingredient in the coating that is only slightly soluble in water.

If, however, the DMSO content in the coating solution is high enough for the amount of DMSO remaining in the coating to be able to dissolve both paclitaxel and the binder, an unstructured clear coating is formed. The optional addition and/or optimization of the DMSO content in the coating solution thus allow the targeted formation of only slightly soluble nano-scale and/or micro-scale active ingredient structures in the coating.

By using the coating solution according to the invention a coated balloon catheter with a tightly adhesive coating can be produced from an anti-proliferative active ingredient such as paclitaxel and a mechanically very stable binder matrix made of PEG-PVA copolymer and optionally shellac or a shellac derivative, from which paclitaxel, in the form of nanoscopic and/or microscopic particles, depending on the case of application, can be deliberately transferred at the site of stenosis to the vascular wall by deflating the balloon, and be released there by diffusion into the vascular tissue. The duration of release and thus the cytostatic effect of paclitaxel increases with the size and crystallinity of the active ingredient particles and can therefore be influenced in particular by correspondingly changing the composition of the coating solution.

Further features and advantages of the invention will arise from the below description of a preferred exemplary embodiment with respect to the attached drawing. However, the exemplary embodiment is not to be considered as limiting.

### DESCRIPTION OF THE DRAWING

FIG. 1 shows an electron microscopic image of a paclitaxel/PEG-PVA/shellac (2:1:0.5) coating in the composition magnified 500 times, obtained from a coating solution without DMSO; and

FIG. 2 shows an electron microscopic image of a paclitaxel/PEG-PVA/shellac coating magnified 500 times, having a PEG-PVA to paclitaxel ratio of 1:2, obtained from a coating solution additionally containing 3% DMSO.

### PRODUCTION OF A COATING SOLUTION

### Example 1

First, 25 mg of a PEG-PVA graft copolymer was dissolved in 2 ml water. The PEG content of the PEG-PVA graft polymer was 25 mole percent. A homogeneous polymer solution was formed by gradually adding 8 ml ethanol in fine doses. The anti-proliferative active ingredient paclitaxel was provided in an amount of 50 mg as a solid and added while exposed to ultrasound. Then, a percentage of 12.5 mg of an ammonium salt of shellac was added to the solution. A clear solution with a solid content of 8.75 mg/ml solvent and an ethanol:water volume ratio of 80:20 was obtained.

### Example 2

As described in Example 1, 25 mg of a PEG-PVA graft copolymer was dissolved in 2 ml water. The PEG content of the PEG-PVA graft polymer was 25 mole percent. A homogeneous polymer solution was formed by gradually adding 8 ml ethanol in fine doses. The anti-proliferative active ingredient paclitaxel was provided in an amount of 50 mg as a solid and added while exposed to ultrasound. In addition, 0.3 ml DMSO was added to this solution. A clear solution with a solid content of 7.5 mg/ml solvent and an ethanol:water volume ratio of 80:20 and a DMSO content of additively 3% relative to the total volume of ethanol and water was obtained. Solution experiments showed that the water content of the coating solution can be increased to a maximum of 40 volume percent without paclitaxel precipitating from the solution.

### Example 3

As described in Example 2, a solution of 25 mg of a PEG-PVA graft copolymer in 2 ml water was produced. The PEG content of the PEG-PVA graft polymer was 25 mole percent. A homogeneous polymer solution was formed by gradually adding 8 ml ethanol in fine doses. The anti-proliferative active ingredient paclitaxel was provided in an amount of 50 mg as a solid and added while exposed to ultrasound. A stable, clear solution with a solid content of 7.5 mg/ml solvent and an ethanol:water volume ratio of 80:20 was obtained. The solution did not contain DMSO.

Coating Experiments

A smooth-walled balloon of a commercially available balloon catheter was coated with a coating solution according to Example 1. The coating solution was applied onto the surface of the balloon catheter by spray coating.

Following the application of the coating solution the balloon's surface was dried by warm air at a temperature between 30 and 60° C. Drying left a tightly adhesive and mechanically stable coating substantially made of nanoscopic paclitaxel in the form of needle-like particles which were embedded in a binder matrix made of the PEG-PVA copolymer and shellac.

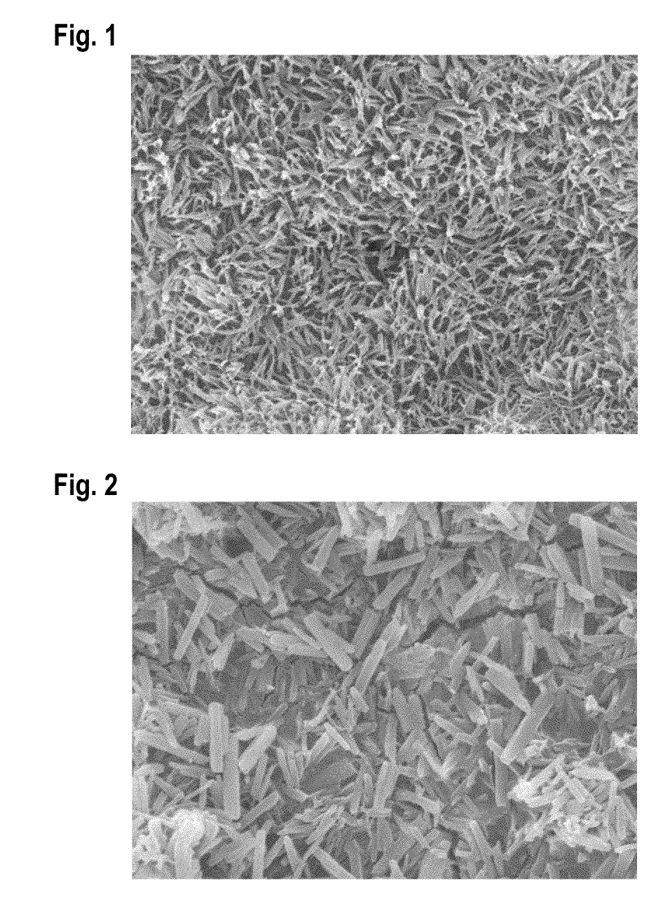

The electron microscopic image of FIG. 1 of a coating produced by using the coating solution of the invention pursuant to Example 1 shows that there is a coating present in the form of needle-like nanoscopic paclitaxel particles linked to each other like in a network.

Drying of the coating solution pursuant to Example 2, without addition of shellac but with a percentage of additively 3% DMSO, on the balloon's surface also resulted in a stable and tightly adhesive coating containing needle-like paclitaxel microparticles in a binder matrix made of PEG-PVA copolymer.

FIG. 2 shows an electron microscopic image of the coating obtained by using the coating solution pursuant to Example 2 and containing DMSO but no shellac. Microscopically, the coating has a needle-like structure and exhibits a distinct phase separation.

The coating solution pursuant to Example 3 resulted in a coating with a nanoscopic structure of the active ingredient particles, similar to the coating obtained by using the coating solution pursuant to Example 1.

Loading of the surface of the balloon catheter with paclitaxel can be freely set by using the method of the invention. Both the resistance of the coating and the structurization of the active ingredient can be further controlled by the ratio of the active ingredient to the water-soluble binder.

The balloon catheter thus produced is particularly suitable for treatment of residual stenosis after vascular dilatation or stent placement.

1. A coated balloon catheter comprising a catheter substrate and a coating on the catheter substrate, the coating comprising a pharmaceutically active ingredient embedded in a binder matrix, wherein the binder matrix consists of a polyethylene glycol-polyvinyl alcohol copolymer (PEG-PVA copolymer) and optionally shellac or a shellac derivative.

2. The balloon catheter according to claim 1, wherein the active ingredient is selected from the group consisting of anti-proliferative, immunosuppressive, anti-inflammatory, anti-phlogistic, anti-hyperplastic, anti-neoplastic, anti-mitotic, cytostatic, cytotoxic, anti-angiogenic, anti-restenotic, microtubule inhibiting, anti-migrative and/or anti-thrombotic active ingredients, in particular cortisone, everolimus, biolimus, tacrolimus, paclitaxel and/or rapamycin.

3. The balloon catheter according to claim 1, wherein the PEG-PVA copo-lymer has a PEG content of 15 to 30 mole percent and a PVA content of 70 to 85 mole percent.

4. The balloon catheter according to claim 1, wherein the PEG-PVA copolymer has an average molar mass Mₙ of 30,000 to 60,000 g/mole.

5. The balloon catheter according to claim 1, wherein the weight ratio of the active ingredient to the PEG-PVA copolymer in the coating is in a range of from about 10:1 to 1:2.

6. The balloon catheter according to claim 1, wherein the weight ratio of the active ingredient to the PEG-PVA copolymer in the coating is in a range of from about 2:1 to 1.1.

7. The balloon catheter according to claim 1, wherein the binder matrix contains shellac or a shellac derivative, and wherein the weight ratio of shellac or the shellac derivative to the PEG-PVA copolymer in the coating is less than or equal to 1:1.

8. The balloon catheter according to claim 1, wherein the loading of the catheter substrate with the active ingredient is in a range of from 0.5 μg/mm² to 10 μg/mm² relative to the outer surface of the substrate when expanded.

9. The balloon catheter according to claim 1, wherein weight of the coating applied onto the catheter substrate is in a range of from 0.75 μg/mm² to 20 μg/mm².

10. The balloon catheter according claim 1, wherein the active ingredient is in the form of active ingredient particles embedded in the binder matrix.

11. The balloon catheter according claim 1, wherein the active ingredient particles are present in the form of nanoscopic and/or microscopic particles.

12. A composition for coating of a balloon catheter, the composition comprising a pharmaceutically active ingredient and a binder dissolved in a solvent mixture, wherein the binder consists of a PEG-PVA copolymer and optionally shellac or a shellac derivative, and wherein the solvent mixture comprises water and at least one additional organic solvent fully miscible with water.

13. The composition according to claim 12, wherein the composition contains water in a percentage of 5 to 40 volume percent.

14. The composition according to claim 12, wherein the solvent mixture additionally contains DMSO in a percentage of up 10 volume percent, relative to the total volume of water and the additional organic solvent.

15. The composition according to claim 12, wherein the additional organic solvent comprises at least one of ethanol and methanol.

16. The composition according to claim 12, wherein the concentration of the active ingredient in the coating composition is up to 25 mg/ml.

17. The composition according to claim 12, wherein the solids content in the coating solution is up to 150 mg/ml.

18. The composition according to claim 12, consisting of
the pharmaceutically active ingredient at a concentration of up to 25 mg/ml, wherein the pharmaceutically active ingredient is selected from the group consisting of an anti-proliferative compound, and immunosuppressive compound and mixtures thereof;
the PEG-PVA copolymer at a concentration of 1 to 50 mg/ml,
a water-soluble shellac derivative at a concentration of 0 to 50 mg/ml;
water in an amount of 5 to 40 volume percent,
at least one of methanol and ethanol in an amount of 60 to 94 volume percent; and
DMSO in a percentage of 0 to 10 volume percent, preferably 0.5 to 10 volume percent.

19. A method for the production of a composition of claim 12, comprising the steps of:
a) providing an aqueous solution of a PEG-PVA copolymer and optionally a water-soluble shellac derivative;
b) gradually adding at least one additional organic solvent indefinitely miscible with water to the aqueous solution of step a) to form an aqueous-organic solution;
c) mixing the aqueous-organic solution with a pharmaceutically active ingredient and homogenizing the mixture to form a coating solution containing the active ingredient;
d) optionally adding DMSO in a percentage of up to 10 volume percent to the coating solution containing the active ingredient of step c) relative to the total volume of water and organic solvent.

20. A method for the production of a coated balloon catheter according to claim 1, comprising the steps of:
a) providing an aqueous solution of a PEG-PVA copolymer and optionally a water-soluble shellac derivative;
b) gradually adding at least one additional organic solvent indefinitely miscible with water to the aqueous solution of step a) to form an aqueous-organic solution;
c) mixing the aqueous-organic solution with a pharmaceutically active ingredient and homogenizing the mixture to form a coating solution containing the active ingredient;
d) optionally adding DMSO in a percentage of up to 10 volume percent to the coating solution containing the active ingredient of step c) relative to the total volume of water and organic solvent; and
e) applying the coating solution containing the active ingredient of step c) or d) onto a surface of a balloon catheter and drying the coating solution to form a coated balloon catheter.
